(19) United States
(12) Patent Application Publication
(10) Pub. No.: US 2023/0101683 A1
(43) Pub. Date: Mar. 30, 2023

(54) FLUID MANAGEMENT SYSTEM

(71) Applicant: Meditrina, Inc., San Jose, CA (US)

(72) Inventor: Daniel TRUCKAI, Saratoga, CA (US)

(73) Assignee: Meditrina, Inc., San Jose, CA (US)

(21) Appl. No.: 17/933,711

(22) Filed: Sep. 20, 2022

Related U.S. Application Data
(60) Provisional application No. 63/261,721, filed on Sep. 27, 2021.

(51) Int. Cl. A61M 5/142; A61M 5/14; A61M 5/172

(52) U.S. Cl. CPC A61M 5/14228; A61M 5/1413; A61M 5/172; A61M 2205/3331

(57) ABSTRACT

Fluid management systems used, for example, in endoscopic procedures.

### RELATED APPLICATIONS

This application is a non-provisional of U.S. Provisional Application No. 63/261,721 filed Sep. 27, 2021, the entirety of which is incorporated by reference herein.

### BACKGROUND OF THE INVENTION

Field of the Invention The present invention relates to a fluid management system of the type used, for example, in endoscopic procedures.

Surgical fluid management systems typically deliver a fluid, such as saline, to a targeted working space or body cavity to provide access and visibility to the physician performing a procedure in the space. The fluid usually provides pressure sufficient to “open” the space (i.e., create a working space for the procedure) and additionally will usually flush blood and debris from the space. Typically, the fluid management system includes a control system for maintaining a preset fluid pressure in a working space.

Surgical fluid management systems are often inconvenient to use and difficult to monitor. Further, fluid management systems often have control systems that are unable to accurately measure pressure in a working space when the patient and the fluid management console are at different elevations. It would therefore be beneficial to provide improved surgical fluid management systems that overcome at least some of these shortcomings.

### SUMMARY OF THE INVENTION

In general, the fluid management system includes a roll stand carrying a control unit and controller, a disposable or multiple-use cassette that is adapted to pressurize a pressure cuff carrying a saline bag, a drape pump, a collection reservoir, and a display for displaying operating parameters of the fluid management system. During operation, the system can calculate pressure in the working space based on fluid pressure in the cassette tubing and provide for inflow and outflow control to maintain a desired pressure in the working space or adjust other operating parameters. Other features, objects, and advantages will be apparent from the description and drawings and from the claims.

The present invention provides improved fluid management systems and methods for their use. In particular, the present invention provides a disposable or multiple-use cassette, with first and second flexible tubing loops therein for coupling to first and second roller pumps. The first tubing loop is used for pressurizing a pressure cuff for delivering fluid from a fluid source to a patient. The second tubing loop may be used for regulating fluid outflows from the patient and delivering the fluid outflows to a disposal waste reservoir. The fluid management systems may also be configured to alert the user when the cassette has been successfully loaded or, conversely, when the cassette has not been successfully loaded. Further capabilities include sensing conditions of the fluid, in particular, positive pressures in the first tubing loop in the cassette. Automatic locking capabilities for locking the cassette in place may also be provided by a motor and control mechanism carried by the control unit.

In other specific embodiments, the cassette may further comprise a flexible membrane on a sensing window on at least one of the first and second tubing loops. The at least one sensing window will usually be positioned to align with a pressure or force sensor on the control unit the cassette is locked into place on the control unit. In an exemplary embodiment, the membrane of the sensing window comprises a thin resilient element overlying an interior chamber in a housing that communicates with the lumen in a tubing loop carried by the cassette.

### BRIEF DESCRIPTION OF THE DRAWINGS

FIG. 1 is a perspective view of a fluid management system including a roll stand with a control unit and peristaltic pumps, a detachable cassette, a pressure cuff and saline bag, a drape pump, a waste collection reservoir, and a tubing set.

FIG. 2 illustrates an enlarged view of the control unit and peristaltic pumps of FIG. 1 and further shows a sliding plate for locking the cassette in place and a pressure sensing mechanism.

FIG. 3 illustrates the front side of the cassette of FIG. 1.

FIG. 4 illustrates the backside of the cassette of FIG. 3 showing first and second tubing loops and a flexible membrane of chamber the fluid flow path of the cassette adapted to interface with a pressure sensing mechanism in the control unit of FIG. 2.

FIG. 5 is a schematic view of the components of the fluid management system of FIG. 1.

FIG. 6 is a perspective view of a flow regulator of the system that is adapted to clamp onto flexible outflow tubing.

### DETAILED DESCRIPTION OF THE INVENTION

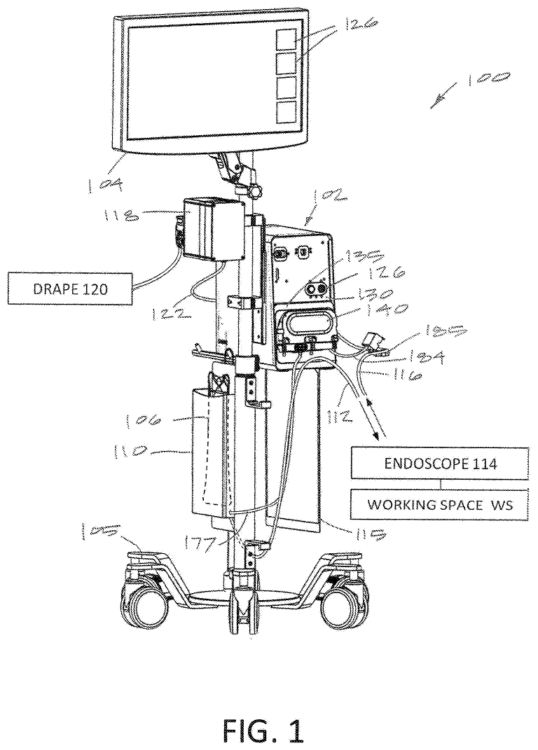

FIG. 1 illustrates a fluid management system 100 of the invention, which includes a control unit 102 and video display 104 mounted on a roll stand 105. The fluid management system 100 is used in endoscopic procedures, which can be a gynecology procedure, a urology surgery or an arthroscopic surgery, to provide inflows and outflows of a pressurized fluid to and from a working space or body cavity. The fluid is typically saline solution and can be delivered through an endoscope or other device to provide and maintain a preset pressure level within the working space. In the system shown in FIG. 1, the fluid is delivered from a fluid source comprising a flexible saline bag 106, which is carried within a pressure cuff 110 as is known in the art. The pressure cuff 110 consists of a flexible structure with an inflatable chamber that surrounds the saline bag 106 where inflation of the pressure cuff 110 compresses the saline bag 106 to cause a fluid flow therefrom through an inflow tubing 112 that is typically coupled to an endoscope 114.

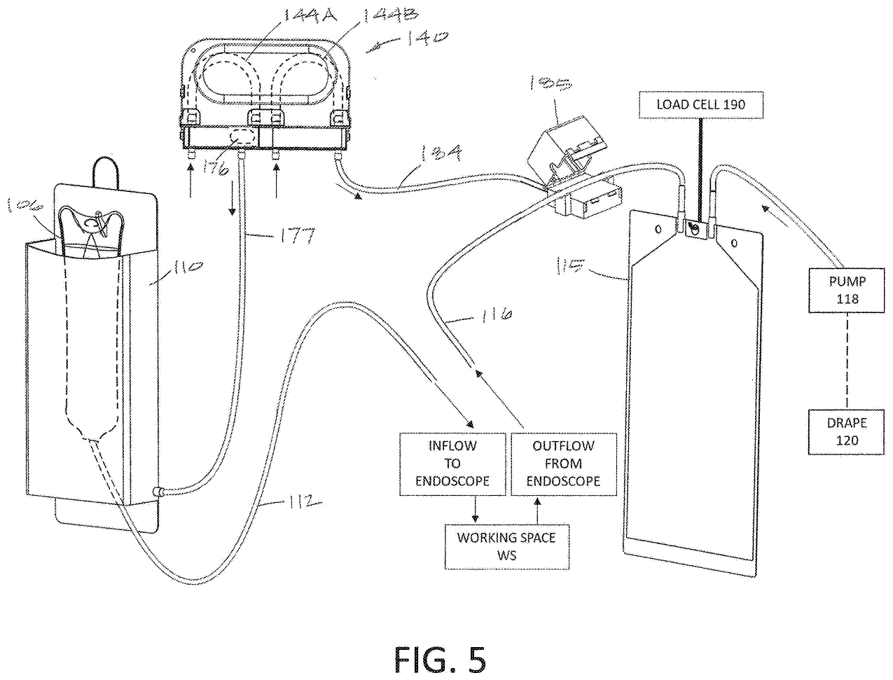

As also can be understood from in FIGS. 1 and 5, the roll stand 105 further carries a waste collection reservoir 115, which is adapted to receive outflows from the working space WS through outflow tubing 116 (FIG. 5) that is coupled to an endoscope or other device. Further, the roll stand 105 carries an independent drape pump 118 (see FIG. 1) for removing fluid from a surgical drape 120 that may be used in a surgical procedure. The drape pump 118 carries fluid from the drape 120 through waste tubing 122 to the waste collection reservoir 115 (FIG. 5). As will be described below, a processor or controller 125 (FIG. 5) in the control unit 102 is configured to sense the weight of fluid in the collection reservoir 115 from which fluid deficits can be calculated and which is important for certain surgical procedures.

In FIG. 1, one variation of a control unit 102 is coupled to a type of video display 104 that comprises a touch screen. In this variation, various icons 126 on the touch screen display 104 are adapted for selecting a target pressure in the working space, a fluid inflow rate, flush, image capture with the endoscope, light intensity of LEDs carried by the endoscope, and/or other modes of operation. It should be appreciated that a smaller touchscreen can be integrated into the control unit 102 itself. Alternatively, an endoscope coupled to the control unit 102 has a control pad with controls buttons for adjusting any operating parameter of the system, such as set pressure, inflow rate, image capture, light intensity, flush, and the like. The touch screen or an actuator on the control unit 102 can be used to unlock or lock the cassette on the control unit as will be described further below.

The video display 104 also is configured to receive data from the controller 125 to continuously display the pressure in the working space, the fluid deficit as will be further described below, the volume of saline remaining in the saline bag 106, the fluid inflow rate. In some variations, the endoscope 114 may be coupled to the controller, and LED light intensity may be displayed on the video display 104 as well as other operating parameters of the endoscope. The endoscope 114 can be coupled to the control unit at connector 126 in the control unit 102.

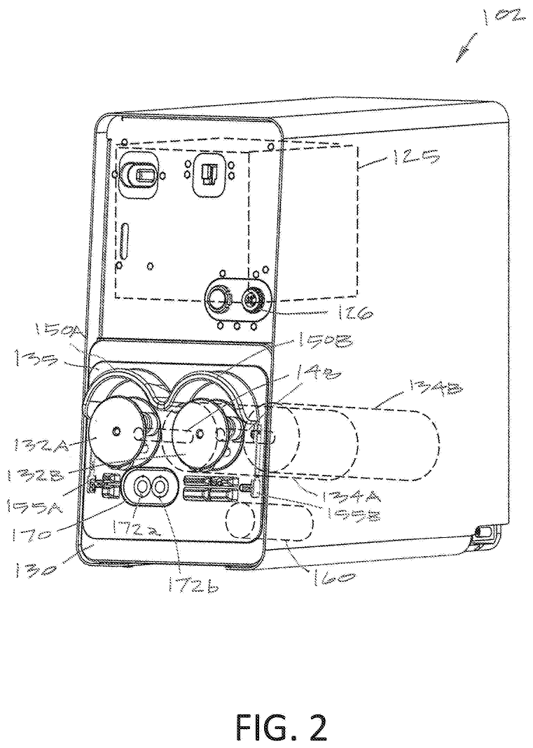

Now referring to FIGS. 1 and 2, the control unit 102 has a front surface 130 that carries first and second peristaltic roller pumps 132A and 132B with corresponding motors 134A and 134B in the interior of the control unit 102 (see FIG. 2). The peristaltic roller pumps 132A and 132B extend through a slidable base plate 135 in the front surface 130 of the control unit 102. As can be seen in FIG. 1, a removable cassette 140 is provided that is adapted for looking in position in the base plate 135 (FIG. 2) to be engaged by the first and second roller pumps 132A and 132B.

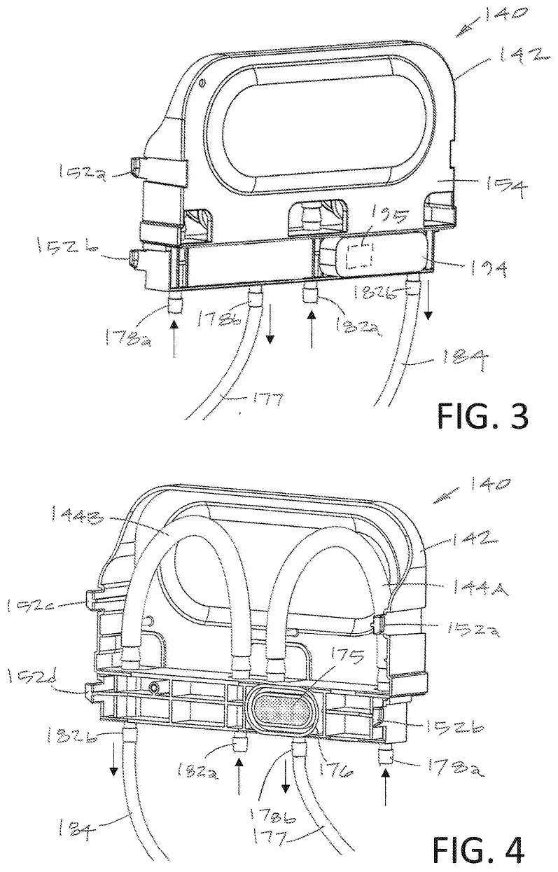

Referring to FIGS. 3-4, the cassette 140 includes a plastic molded housing or body 142 that carries portions of a tubing set, and more particularly two loops of a flexible tubing 144A and 144B as in known in the art. The tubing loops are typically a flexible polymer material having a diameter ranging between about ¼″ to ½″ and are adapted to cooperate with the first and second pumps 132A and 132B (see FIGS. 1 and 2). The tubing loops 144A and 144B in the cassette 140 (see FIG. 4) extend in a semicircular arc of at least 90° or at least 120° in the plane of the cassette, where the plane of the cassette is adapted to align with the first and second roller pumps 132A and 132B. As can be understood from FIGS. 2 and 4, the plane of the tubing loops is perpendicular to the axis of each shaft 148 of the pump motors 134A and 134B.

The system 100 and control unit 102 includes a mechanism for locking the cassette 140 in place. Referring to FIGS. 2 and 4, it can be understood that the tubing loops 144A and 144B within the cassette 140 are adapted to be inserted between the roller pumps 132A and 132B and the arcuate structure or eyebrows 150A and 150B (FIG. 2). As can be seen in FIG. 2, the slidable base plate 135 carries the eyebrows 150A and 150B that are adapted to contact and press the tubing loops 144A and 144B against the first and second roller pumps 132A and 132B. Referring to FIGS. 3 and 4, it can be seen that the cassette 140 has four short leg elements 152a-152d extending away from the front face 154 of the cassette 140. In FIG. 2, it can be seen that two sensor boards 155a and 155b are provided on either side of the base plate 135 of the control unit 102. Each sensor board 155a and 155b carries optical sensors (not shown) that sense reflectance of the legs 152a-152d of the cassette 140 when pushed into place against the base plate 135. In other words, as the cassette 140 is pushed inwardly to contact the front surface of the base plate, the four optical sensors detect reflectance of the four legs 152a-152d which then activates a locking motor 160, which in turn moves the base plate 135 in a downward direction to press the eyebrows 150A and 150B against the tubing loops 144A and 144B so that the first and second roller pumps 132A and 132B properly engage the tubing loops. The downward movement of the base plate 135 and eyebrows 150A and 150B then reach a stop position to lock the cassette 140 in place.

As described above, the system allows for precise control of fluid flows and fluid pressure in the working space, which is next described in more detail. Referring again to FIG. 2, it can be seen that the front surface 130 of the control unit 102 carries a pressure sensor mechanism 170 which in a variation includes two pressure sensors 172a and 172b. The first and second pressure sensors 172a and 172b are provided for redundancy. The sensor mechanism 170 is adapted to contact a flexible membrane 175 carried by the cassette 140 (see FIG. 4). It can be understood from FIGS. 2 and 4 that the cassette sensor membrane 175 is disposed on a side of an air chamber 176 in the cassette 140 that communicates with pressure cuff tubing 177. The membrane 175 is adapted to flex inwardly and outwardly depending on pressure of the air in the chamber 176 and the lumen of the tubing loop 144A. Referring to FIGS. 2-4, actuation of the first pump 132A causes air to be suctioned through port 178a in the cassette 140, which communicates with tubing loop 144A, air chamber 176, and the port 178b, which in turn is coupled to the pressure cuff tubing 177, which extends to the pressure cuff 110. The sensor mechanism 170 can then sense pressure in the pressure cuff tubing 177, which in turn measures pressure in the pressure cuff 110, the saline bag 106 and the working space WS, which are all correlated. The controller 125 then can use algorithms to calculate the fluid pressure in the working space WS and then can maintain or modulate pressure in the pressure cuff 110 and working space WS by controlling roller pump 132A.

In one variation, the fluid management system 100 has a controller 125 that is adapted to alter the pressure applied to the pressure cuff 110 that is dependent on the volume of saline remaining in the saline bag 106. It can be understood that as the saline in the bag 106 has a greatly reduced volume, pressure in the cuff 110 may not cause the same saline inflow rate as when the saline bag has a full volume. Therefore, the controller 125 can use a look-up table or similar mechanism to correlate pressure provided by the roller pump with the volume of saline in the saline bag. The pressure cuff 110 can be configured with a sensor mechanism coupled to the controller 125 for sensing the volume of fluid in the saline bag 106, wherein the sensor system comprises at least one of a weight sensor, an ultrasound sensor, an optical sensor or an electrical sensor wherein the controller 125 is responsive to signals from such a sensor mechanism to adjust operation of the roller pump to provide a flow of air into the cuff 110 the volume of saline is at any particular level. As described above, the positive pressure in the cuff 110 is used to provide fluid flows into a working space WS from the saline bag 106. The roller pump 132A can apply negative pressure to collapse the pressure cuff 110, for example, to change the saline bags or too rapidly reduce pressure in the working space WS.

In another aspect, the system 100 and controller 125 provides a mechanism or flow regulator for controlling fluid outflows from the working space WS. Referring to FIGS. 2, 3 and 4, the second peristaltic roller pump 132B engages the second tubing loop 144B in the cassette 140. Actuation of second roller pump 132B causes air to be suctioned through port 182a in the cassette 140, which communicates with tubing loop 144B with the air exiting the tubing loop 144B through port 182b into clamp tubing 184, which in turn is coupled to the clamp valve 185 as shown in FIGS. 2 and 5. The pump 132B then can be used to actuate an actuator in the clamp valve 185 to pinch the outflow tubing 116 that is clamped into the clamp valve 185. The controller 125 is adapted to control the second roller pump 132B to apply positive or negative pressure to the clamp valve to decrease or increase flows through the outflow tubing 116.

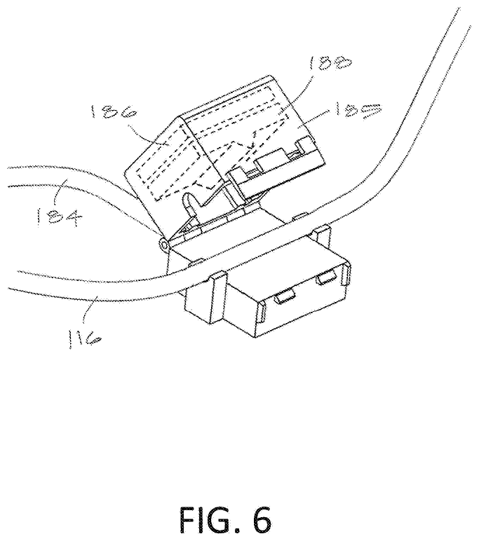

In one variation shown in FIG. 6, the clamp valve 185 includes internal bladder 186, which when expanded by airflow through tubing 184 is adapted to move a sliding pinch element 188 into the flexible outflow tubing 116. It can be understood that the pinch element 188 applies pressure to the exterior of the flexible outflow tubing 116 and can thereby control to the fluid outflow rate. Thus, the controller 125 can thus control both roller pumps 132A and 132B in response to signals from the pressure sensor mechanism 170 to vary the inflows and/or outflows to maintain a targeted set pressure in the working space or a targeted pressure in combination with a selected inflow or outflow rate.

In another aspect, the system 100 and controller 125 are capable of determining fluid deficit, which is important is some surgical procedures, such as gynecology. As can be understood from FIGS. 1 and 5, the roll stand 105 and control unit 102 carry a load sensor 195 that provides a signal to the controller 125 of the weight of saline collected in collection reservoir 115, which is collected from fluid outflows and well as collected from the drape 120. The fluid deficit monitoring system is calibrated at the start of a procedure by providing the controller 125 with the known volume of saline in the saline bag 106. Another load sensor optionally could be used to monitor the weight of the saline bag 106 and pressure cuff 110. The controller 125 then is configured to calculate the volume of saline inflows to the working space WS and the volume of saline collected in the collection reservoir 115 from which the fluid deficit then is known. In one variation, the fluid deficit is continuously calculated and shown on the display 104. The system 100 further can include audio and/or visual alarms when the fluid deficit reaches a preselected level, and the system can include a shut-down mechanism when the fluid deficit reaches a preselected level.

In another aspect of the invention, referring to FIG. 3, the cassette 140 with tubing loops 144A and 144B can be configured for limited re-use since only air flows through the tubing loops within the cassette 140. In FIG. 3, it can be seen that the front surface 154 of the cassette 140 carries a housing 194 that houses a mechanical counter mechanism 195 that automatically advances the counter each time the cassette is pushed into contact with the front surface 130 of the control unit 102. In another variation, the cassette 140 can carry an identifier such as an RFID identifier that the controller 125 can sense and monitor for the number of uses. In such a variation, the controller 125 then would not operate after a selected number of uses, for example, ten or twelve uses.

Although particular embodiments of the present invention have been described above in detail, it will be understood that this description is merely for purposes of illustration and the above description of the invention is not exhaustive. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A fluid management system, comprising:
a control unit carrying a first peristaltic pump and a second peristaltic pump;
a cassette carrying a first tubing loop and a second tubing loop, each adapted for coupling to the first peristaltic pump and the second peristaltic pump, respectively;
a pressure cuff;
a saline bag carried within the pressure cuff;
wherein the first peristaltic pump and the first tubing loop are adapted to pump air into the pressure cuff to apply pressure to an exterior of the saline bag to cause fluid flows from the saline bag into a working space; and
wherein the second peristaltic pump and the second tubing loop are adapted to pump air into a flow regulator coupled to outflow tubing that is configured to carry fluid outflows from the working space.

2. The fluid management system of claim 1 further comprising a controller for controlling operation of the first peristaltic pump and the second peristaltic pump.

3. The fluid management system of claim 2 wherein the first peristaltic pump responsive to the controller is adapted to increase or decrease pressure in the pressure cuff.

4. The fluid management system of claim 2 wherein the second peristaltic pump responsive to the controller is adapted to increase or decrease pressure in the flow regulator.

5. The fluid management system of claim 2 further comprising a sensor mechanism coupled to the controller for sensing a volume of fluid in the saline bag, wherein the sensor mechanism comprises at least one of a weight sensor, an ultrasound sensor, an optical sensor and an electrical sensor.

6. The fluid management system of claim 5 wherein the controller is responsive to signals from the sensor mechanism is adapted to adjust operation of the first peristaltic pump to provide a selected rate of fluid flow from the saline bag when the volume of fluid in the saline bag is at any volume level.

7. The fluid management system of claim 5 wherein the control unit carries a pressure sensor for sensing pressure in the first tubing loop of the cassette, and the pressure sensor sends pressure signals to the controller.

8. The fluid management system of claim 7 wherein the controller is adapted to maintain a set pressure in the working space in response to the pressure signals.

9. The fluid management system of claim 6 further comprising a collection reservoir adapted to collect fluid after being circulated through the working space.

10. The fluid management system of claim 9 further comprising a surgical drape adapted to collect a volume of fluid lost from the working space.

11. The fluid management system of claim 10 further comprising a drape pump adapted to pump fluid from the surgical drape to the collection reservoir.

12. The fluid management system of claim 11 further comprising a weight sensor adapted to sense a weight of fluid in the collection reservoir.

13. The fluid management system of claim 12 wherein the controller is adapted calculate fluid deficit.

14. The fluid management system of claim 2 wherein the controller is adapted to lock and unlock the cassette from the first peristaltic pump and the second peristaltic pump.
